(19) United States
(12) Patent Application Publication
(10) Pub. No.: US 2022/0053799 A1
(43) Pub. Date: Feb. 24, 2022

(54) POSITIVE LATENCY EFFECTS ON COCCIDIOSIS PREVENTION AND TREATMENT VIA ANIMAL FEED

(71) Applicant: ZIVO Bioscience, Inc., Keego Harbor, MI (US)

(72) Inventors: William P. Pfund, Keego Harbor, MI (US); Amy E. Steffek, Keego Harbor, MI (US); Andrew A. Dahl, Keego Harbor, MI (US)

(73) Assignee: ZIVO Bioscience, Inc., Keego Harbor, MI (US)

(21) Appl. No.: 17/358,953

(22) Filed: Jun. 25, 2021

Related U.S. Application Data
(60) Provisional application No. 63/044,770, filed on Jun. 26, 2020.

(51) Int. Cl. A23K 20/163; A23K 50/75; A61K 31/739; A23K 10/18

(52) U.S. Cl. CPC A23K 20/163; A23K 10/18; A61K 31/739; A23K 50/75

(57) ABSTRACT

An effective treatment method and composition for controlling a variety of diseases including disease in poultry are disclosed. The inventive concept set forth herein provides an improved long-lasting treatment having a positive latency effect for a broad variety of diseases that is easy to administer and cost effective. The disclosed method of treatment utilizes a compound derived from a lipopolysaccharide (LPS) of gram-negative bacteria. By administering the compound early in broiler life, disease prevention and treatment via immune modulation result. The treatment has a lasting effect throughout the entire broiler production period. The composition itself is a natural product and thus has no adverse environmental impact unlike known antibiotic regimens. By providing effective treatment during the stage of life where feed consumption is lowest by volume, costs to the producer are advantageously limited.

### CROSS REFERENCE TO RELATED APPLICATION

This application is a US. Non-provisional patent application of U.S. Provisional Patent Application No. 63/044,770, entitled “Positive Latency Effects on Coccidiosis Prevention and Treatment Via Animal Feed,” filed Jun. 26, 2020, which is herein incorporated by reference in its entirety for all purposes.

### TECHNICAL FIELD

The present invention relates to the use of a bacteria-based compound in the in the prevention and treatment of intestinal tract disease. More particularly, the present invention relates to the use of a compound derived from a lipopolysaccharide (LPS) of a gram-negative bacteria in the prevention and treatment of an intestinal tract disease such as coccidiosis by way of an animal feed regimen specifically administered early in the animal's life. The effective compound may also be derived from a source other than a lipopolysaccharide.

### BACKGROUND OF THE INVENTION

Substantial economic losses in the poultry industry are most often the result of disease. Diseases in flocks often result in reduced production volume or compromised quality of meat. Prevention and treatment of poultry disease adds significantly to poultry production costs. Some estimates place total losses as a result of poultry disease at more than 10% of all production costs.

Of the diseases known to strike poultry flocks, the most common are enteric diseases which include coccidiosis, a disease caused by a parasite, the coccidian protozoa. Annual economic losses due to coccidiosis alone are estimated to exceed $3 billion per year and these costs are expected to increase due to a variety of reasons.

First, coccidiosis prevention today is accomplished mainly through the use of vaccines. A one-time administration of the vaccine is given very early in broiler life and, specifically, on the day of hatch. While this approach has shown some benefit, vaccines are known to suffer from variable effectiveness in controlling the disease over time. Experimentation has shown that a vaccine used in conjunction with a supplement such as a probiotic may improve outcome, but this approach but this approach faces its own challenges.

Second, coccidiosis treatment today is accomplished conventionally through the use of antibiotics and ionophores, both of which are costly. The use of antibiotics and ionophores is under pressure globally for a number of reasons, including environmental concerns related to the emergence of antibiotic-resistant pathogens. Drug resistance to antibiotics, ionophores, and synthetic treatment compounds is increasing largely due to overuse thereby severely compromising the effectiveness of these treatments. Relatively recently the European Union banned sub-therapeutic doses of certain antibiotics for use as feed additives. There has been no approval of new drugs in any of these categories for many years. Synthetic treatment compounds and other chemical agents are known but are not as effective as conventional antibiotics.

Fourth, even if known treatments were still economical and effective, known approaches would still be regarded as unsatisfactory because the medication must be included in the animal's feed for the full duration of its lifespan to be fully effective. This requirement adds significant cost to feed for the entire growout period.

Accordingly, it is desirable to develop a non-antibiotic based treatment of pathogenic infections such as coccidiosis in poultry that needs to only be administered during the earliest stage of animal growth without requiring subsequent treatments. Such an extended latency period without the need for later treatment will provide considerable cost savings in the industry while assuring animal health.

### SUMMARY OF THE INVENTION

The disclosed inventive concept provides an improved long-lasting treatment for a broad variety of diseases for use in both animals and humans. Such diseases in animals may include, but not be limited to coccidiosis, that is easy to administer and cost effective. The disclosed method and composition provide both disease prevention and treatment via immune modulation early in broiler life. By providing effective treatment during the stage of life where feed consumption is lowest by volume, costs to the producer are advantageously limited.

The treatment has a lasting effect throughout the entire broiler growth cycle. The composition itself is a natural product and thusly has no adverse environmental impact unlike antibiotic regimens.

When the disclosed compound derived from a lipopolysaccharide (LPS) of gram-negative bacteria is administered to the animal by way of poultry feed, drinking water, or both, the actives of the compound mitigate the effects of coccidiosis well after withdrawal of the active material even in the presence of ongoing coccidiosis exposure. Thus, the approach of the disclosed inventive concept stands in sharp contrast to known and commonly used treatments for which the effect is dependent on the on-going presence of the active agent in the feed. The disclosed compound may also have positive latency effect when fed early to bovine, porcine, avian, equine, ovine, lapine, and caprine species.

### DESCRIPTION OF THE DRAWINGS

For a more complete understanding of this invention, reference should now be made to the accompanying figures. As set forth in many of the figures, the designation “No Tx, No Challenge” refers to a test in which no treatment was administered to a subject animal not deliberately infected with coccidiosis. The designation “No Tx, Cocci” refers to a test in which no treatment was administered to a subject animal deliberately infected with coccidiosis. The designation “Anti-cocci, Cocci” refers to a test in which the subject animal was infected with coccidiosis and the animal was administered an anticoccidial.

The designation “ZIVO all rations, Cocci” refers to a test in which the subject animal was infected with coccidiosis and the animal was administered a treatment composition according to the disclosed inventive concept. The designation “ZIVO starter & grower, Cocci” refers to a test in which the subject animal was infected with coccidiosis and the animal was administered a starter diet at 0-21 day of age. The designation “ZIVO starter, Cocci” refers to a test in which the subject animal was infected with coccidiosis and the animal was administered a grower diet at 22-35 days of age. The designation “ZIVO, grower & finisher, Cocci” refers to a test in which the subject animal was infected with coccidiosis and the animal was administered a grower and finisher diet at 36-42 days of age.

The accompanying figures are described as follows:

FIG. 1 is a graph illustrating test subject feed conversion data for Days 0 to 7;

FIG. 2 is a graph illustrating test subject feed conversion data for Days 8 to 14;

FIG. 3 is a graph illustrating test subject feed conversion data for Days 15 to 21;

FIG. 4 is a graph illustrating test subject feed conversion data for Days 22 to 28;

FIG. 5 is a graph illustrating test subject feed conversion data for Days 29 to 42;

FIG. 6 is a graph illustrating test subject feed conversion data for Days 0 to 42;

FIG. 7 is a graph illustrating test subject lesion scores determined on Day 21;

FIG. 8 is a graph illustrating test subject lesion scores determined on Day 42;

FIG. 9 is a graph illustrating test subject ileum villi cell height on Day 21;

FIG. 10 is a graph illustrating test subject ileum villi cell height on Day 42;

FIG. 11 is a graph illustrating test subject ileum crypt depth on Day 21;

FIG. 12 is a graph illustrating test subject ileum crypt depth on Day 42;

FIG. 13 is a graph illustrating test subject ileum cell height to crypt depth ratio on Day 21;

FIG. 14 is a graph illustrating test subject ileum cell height to crypt depth ratio on Day 42;

FIG. 15 is a graph illustrating test subject Salmonella cecum count on Day 21;

FIG. 16 is a graph illustrating test subject Salmonella cecum count on Day 42;

FIG. 17 is a graph illustrating test subject Clostridium perfringens fecal count on Day 21;

FIG. 18 is a graph illustrating test subject Clostridium perfringens fecal count on Day 42;

FIG. 19 is a graph illustrating test subject E. coli fecal count on Day 21;

FIG. 20 is a graph illustrating test subject E. coli fecal count on Day 42;

FIG. 21 is a graph illustrating test subject duodenum loop oocycst count on Day 21;

FIG. 22 is a graph illustrating test subject duodenum loop oocycst count on Day 42;

FIG. 23 is a graph illustrating test subject mid-gut oocycst count on Day 21;

FIG. 24 is a graph illustrating test subject mid-gut oocycst count on Day 42;

FIG. 25 is a graph illustrating test subject whole cecum oocycst count on Day 21;

FIG. 26 is a graph illustrating test subject whole cecum oocycst count on Day 42;

FIG. 27 is a graph illustrating test subject mortality for Days 0 to 7;

FIG. 28 is a graph illustrating test subject mortality for Days 8 to 14;

FIG. 29 is a graph illustrating test subject mortality for Days 15 to 21;

FIG. 30 is a graph illustrating test subject mortality for Days 22 to 28;

FIG. 31 is a graph illustrating test subject mortality for Days 29 to 42; and

FIG. 32 is a graph illustrating test subject mortality for Days 0 to 42.

### DETAILED DESCRIPTION OF THE PREFERRED EMBODIMENT

In the following description, various operating parameters and components are described for different constructed embodiments. These specific parameters and components are included as examples and are not meant to be limiting. Unless otherwise noted, all technical and scientific terms used herein are to be accorded their common meanings as would be understood by one having ordinary skill in the art.

The method of the disclosed inventive concept proposes the use of a compound comprising an algal biomass as well as related materials including, for example, algal supernatant, symbiont bacteria, bacterial biomass, and bacterial fermentate.

The Compounds Used in Treatment

In general, delivery of the composition is made by oral administration of the active materials mixed into feed or drinking water. The disclosed method of treatment preferably, but not absolutely, utilizes a compound generally derived from a lipopolysaccharide (LPS) of Gram-negative bacteria. By administering the compound early in broiler life, disease prevention and treatment via immune modulation are achieved. As used herein, the term “inhibitor” refers to a molecule that reduces or attenuates the activity induced by another molecule, receptor, cellular structure, or organ. By way of example, a compound that might block the LPS-dependent activation of TLRs, such as but not limited to TLR4, present on the surface of a host immune cell would be regarded as an inhibitor of this particular pathway. Conversely, the term “activator” or “agonist” refers to a molecule that increases or enhances the activity induced by another molecule, receptor, cellular structure, or organ.

As used herein, the term “algal culture” is defined as an algal organism and bacteria (one or more types) that grow together in a liquid medium. Unless expressly stated otherwise, the term “algal biomass” refers to the algal cells and bacterial cells (with the liquid culture medium removed). The “algal biomass” can be wet material or dried material.

Unless expressly stated otherwise, the term “algal supernatant” is defined as the culture medium in which the algal biomass is grown that contains excreted compounds from the algal biomass. Algal supernatant is obtained by growing algal biomass in culture medium for an appropriate length of time and then removing the algal and bacterial cells by filtration and/or centrifugation.

It is known that bacteria of the Variovorax genus and the Rhodobacter genus are metabolically versatile. Variovorax is a Gram-negative aerobic bacterium that can grow under a variety of conditions. It is part of the subclass Proteobacteria and is capable of metabolically utilizing several natural compounds generated by plants or algae. Rhodobacter can grow under a broad variety of conditions, utilizing both photosynthesis and chemosynthesis. Growth can also be achieved under both anaerobic and aerobic conditions. Rhodobacter sphaeroides represents a Gram-negative facultative bacterium and is a member of the α-3 subdivision of the Proteobacteria.

Embodiments of the compound used in the treatment of disease as set forth herein include one or more LPS/Lipid A compounds produced by Gram-negative bacterial strains for use as selective modulators of the TLR signaling pathway, such as the TLR4 pathway. The disclosed inventive concept involves any combination of three fundamental steps: (1) the Gram-negative bacteria produces LPS/Lipid A compounds; (2) the LPS/Lipid compounds modulate TLR4 activity through inhibition or activation; and (3) a downstream effect results in modulated inflammation and recruitment of immune cells of the gut via the modulation of TLR4 signaling, thereby aiding in the treatment of coccidiosis, necrotic enteritis, and other conditions related to gut inflammation.

In an embodiment, the LPS/Lipid A compounds used as selective modulators of the TLR4 signaling pathway are produced from a Variovorax paradoxus strain. The Variovorax paradoxus strain may be a naturally occurring strain.

In another embodiment, the LPS/Lipid A compounds used as selective modulators of the TLR4 signaling pathway are produced from a Rhodobacter sphaeroides strain. Extensive studies have been undertaken regarding the structure and function of Rhodobacter sphaeroides. More focused studies have examined the photosynthetic characteristics of Rhodobacter sphaeroides. It is known that lipopolysaccharides from Rhodobacter sphaeroides are effective TLR4 antagonists in human cells that prevent TLR4-mediated inflammation by blocking LPS/TLR4 signaling. In cells of other species, LPS from Rhodobacter sphaeroides acts as an agonist of the TLR4 pathway. The inventors employed a testing methodology to address multiple immune response mechanisms in poultry to arrive at the conclusion that an LPS compound derived from Rhodobacter sphaeroides proved effective as a coccidiostat in poultry. Initial data suggested modulation by an LPS-like molecule, it was not until specific testing directed to Rhodobacter sphaeroides revealed the effectiveness of this bacterium in the treatment of disease, such as in the treatment of coccidiosis in poultry. Research further showed that combining a TLR4 inhibitor with an activator of TLR2 (such as lipoprotein from Gram-negative bacteria) provides an anti-coccidiosis effect.

Accordingly, embodiments of the compound used in the treatment of disease according to the present disclosure are directed to one or more LPS/Lipid A compounds produced by a Gram-negative bacterial strain of the group Variovorax or the group Rhodobacter for use as selective modulators of the TLR4 signaling pathway. A specific embodiment of the disclosed inventive concept is directed to the use of an LPS/Lipid A compound used as a selective modulator of the TLR4 signaling pathway produced from the Variovorax paradoxus strain and the Rhodobacter sphaeroides strain.

The LPS/Lipid A compound employed herein may be obtained from the Variovorax paradoxus strain and/or the Rhodobacter sphaeroides strain by any suitable method, but in specific embodiments they are extracted using standard multi-step LPS extraction protocols, such as: (1) extracting freeze-dried bacteria with a solution of phenol/guanidine thiocyanate and collecting the water layer for freeze-drying; (2) resolubilizing the freeze-dried fraction in water; (3) ultrafiltration of the solubilized fraction to remove low molecular weight substances and salts; (4) affinity purifying the high-molecular weight fraction using a polymyxin B resin column such as Affi-prep polymyxin matrix material (Bio-Rad), from which an active fraction is eluted with 1 deoxycholate and, optionally; (5) performing additional purification using size-exclusion chromatography.

In some examples, multiple types of LPS extraction protocols are employed to obtain an LPS compound from the bacteria, and extraction procedures may be performed more than once. Once the LPS compound is extracted and purified from the bacteria, the Lipid A fraction may be prepared by acid hydrolysis or other suitable technique.

The one or more LPS/Lipid A compounds derived from Gram-negative bacterial strains, such as Variovorax paradoxus or Rhodobacter sphaeroides, may selectively modulate the TLR4 signaling pathway to modulate inflammatory responses and to improve immune health in a variety of uses and applications. In an embodiment, the LPS/Lipid A compound derived from Variovorax paradoxus or Rhodobacter sphaeroides may be incorporated within an grain-based feed to improve gut health of poultry.

The disclosed LPS/Lipid A compound derived from Variovorax paradoxus or Rhodobacter sphaeroides may be used to improve the health of poultry through a variety of mechanisms. For example, if acting as an inhibitor, the LPS/Lipid A compound may protect against internal inflammation in poultry by negatively regulating inflammatory mediators via the downregulation of TLR4 expression and the downstream inhibition of NF-kappa B activation in a typical inflammatory cascade. In another example, the LPS/Lipid A compound may inhibit the activation of TLR4 in poultry by interfering with cysteine residue-mediated receptor dimerization. In yet another example, the LPS/Lipid A compound may inhibit the ability of non-infectious and infectious stimuli to interact with TLR4 and trigger a pro-inflammatory response, thereby improving poultry gut integrity. Alternatively, if working as an agonist of the TLR4 pathway, LPS/Lipid A compound may prime the immune system to better response to invading pathogens by recruiting specific disease fighting immune cells to intestinal tissues in advance of a disease challenge thereby accelerating and heightening the immune response to any subsequent pathogen exposure.

Specific Treatment Compounds

The disclosed treatment compounds are based on one or more fresh water algal biomasses including bacterial strains as discussed above. More particularly, the algal biomass may include the Gram-negative such as Variovorax paradoxus strain or Gram-negative Rhodobacter sphaeroides strain.

Four treatment compounds are presented and considered. The compounds share the common characteristic of the algal biomass referenced above and are used in animal treatment. The algal biomass-based products are fed to animals in a formulated diet such as a corn or corn-soybean meal (SBM) diet or are delivered in drinking water. As noted, the specific treatment compositions include “ZIVO—all rations,” ZIVO starter & grower,” “ZIVO—starter,” and “ZIVO—grower & finisher.”

In all of is variations the ZIVO treatment compound is fresh water algal biomass containing Gram-negative bacteria provided as animal feed in combination of a feed additive, such as soy oil, preferably though not exclusively at a ratio of two parts soil oil to one part algal biomass. Once the biomass and feed additive are combined to the preferred premix level, the combined batch is poured or administered evenly into a ribbon mixer containing finished feed. The combined batch is preferably provided in an amount of between about 0.5 lbs. composition per ton of finished feed to about 11.0 lbs. composition per ton of finished feed, is more preferably provided in an amount of between about 1.0 lbs. composition per ton of finished feed to about 5.0 lbs. composition per ton of finished feed, and is most preferably provided in an amount of between about 3.0 lbs. composition per ton of finished feed to about 4.0 lbs. composition per ton of finished feed. The ideal suggested and non-limiting ratio is about 3.5 lbs. composition per ton of finished feed.

Studies

Studies were undertaken to determine the response and efficacy of the various treatment compounds. Pellet feed was employed for delivery of the ZIVO treatment compound using a corn-soybean diet type commercial ration formulation. Non-limiting examples of a method for preventing and treating disease in poultry is set forth. It is to be understood that the following method is not intended as being the sole treatment method but is only exemplary. For example, the compound of the disclosed invention may be provided to the animals in water either exclusively or in combination with the addition to dry feed.

Study Treatment Method

A total of 2,184 mixed sex broiler chicks were obtained within twelve hours of hatching from fecal contaminated flocks at a commercial hatchery on Day 0 (hatch and placement day). A number of mixed-sex broiler chicks (50:50 sex ratio) were randomly assigned on Day 0 by individual weights to one of several test group pens, each with replicates. Only antibiotic-free birds were sourced, and no coccidiosis vaccine was administered at the hatchery or at any time during the study. Chicks were evaluated upon receipt for signs of disease or other complications that could affect study outcome. Weak birds were humanely sacrificed. Birds were not replaced during the study. Overall, administration of the inventive composition with the feed produced good results when given during the Starter and Grower phase (0-28 days), produced better results when administered during the Starter phase (0-14 days), and produced the best results when administered during the Grower and Finisher phase (0-7 days).

Following examination, chicks were weighed and allocated to pens for the various treatment groups using a randomized block design. Weight distribution across the treatment groups was assessed prior to feeding by comparing the individual test groups' standard deviations of the mean against that of the control group. Weight distribution across the groups was considered acceptable for this study when differences between control and test groups were within one standard deviation.

Treatment Groups—Treatment groups, the levels of test material, the number of replicates, the number of bird replicates, and the routes of administration were established as follows.

| | | | Treatment | Route | | |
|---|---|---|---|---|---|---|
| | | Cocci | Substance | of | | Birds per |
| Trt. | Treatment Description^{2,3,4} | Challenge | and Level | Admin | Reps | Replicate |
| 1 | Unchallenged/No Treatment | No | None | Feed | 12 | 26 |
| | Control | | | Pellets¹ | | |
| 2 | Challenged/No Treatment | Yes | None | Feed | 12 | 26 |
| | Control | | | Pellets¹ | | |
| 3 | Antibiotic Control | Yes | The anti-coccidia drug | Feed | 12 | 26 |
| | | | Coban ® (Elanco) in | Pellets¹ | | |
| | | | feed at 90 g/ton | | | |
| 4 | Algae Biomass-All Rations | Yes | 3.5 lb/ton of feed | Feed | 12 | 26 |
| | | | (all rations) | Pellets¹ | | |
| 5 | Algae Biomass-Starter & | Yes | 3.5 lb/ton of feed in | Feed | 12 | 26 |
| | Grower | | STARTER & | Pellets¹ | | |
| | | | GROWER | | | |
| | | | RATIONS ONLY | | | |
| 6 | Algae Biomass-Starter only | Yes | 3.5 lb/ton of feed in | Feed | 12 | 26 |
| | | | STARTER RATION | Pellets¹ | | |
| | | | ONLY | | | |
| 7 | Algae Biomass-Grower & | Yes | 3.5 lb/ton of feed in | Feed | 12 | 26 |
| | Finisher Only | | GROWER & FINISHER | Pellets¹ | | |
| | | | RATIONS ONLY | | | |

¹Corn and SBM rations, with normal nutritional formulations.
²ZIVO product is unique fresh-water algae product without harm to feed milling and live production personnel.
³No Coccidiostat or ABF (Antibiotic Free Products) administered during the entire study. One control antibiotic and four test materials were fed to the birds.
⁴No Coccidiosis-Vaccine was administered at the hatchery or during the course of this study.

All birds received nutritionally adequate food or drink compounds. Birds were fed their respective treatment diets ad libitum from day of hatch to 42 days of age, the typical average market age of broiler chickens in US. Birds were raised on built-up litter to further mimic stress conditions typically experienced in poultry production.

Diets were weighed at the beginning of each formulation period and fed in three phases: starter diet (0-21 days of age), grower diet (22-35 days of age) and finisher diet (36-42 days of age). Diets were fed for the entire study duration as pellets. All treatment compound diets were offered ad libitum without restrictions to full-fed consumption, except for an 8-hour fasting period for cocci-inoculated birds prior to cocci-challenge on Day 7.

On Day 7 and 7-days of age (Trial Day 0=hatch and placement day), adequate feed was precisely weighed, provided to consume at the rate of 100% fill-capacity on average for all birds. This was be determined by measuring the quantity of feed consumed within a 24-hr period the day before for each pen. Also on Day 7 all birds in the challenged groups received oocyst-inoculated sustenance containing a mixture of Eimeria acervulina, Eimeria maxima, and Eimeria tenella. Particularly, the birds received sustenance containing a mixture 100,000 oocysts per bird of E. acervulina, 50,000 oocysts per bird of E. maxima, and 75,000 oocysts per bird of E. tenella.

Cocci-Challenge Model —. All challenge organisms were mixed in the Starter Feed using a 50# mixer with a thorough mix running time of about ten minutes. Prior to the challenge, all cocci-inoculated birds were starved for eight hours. Inoculated feed was provided to the birds. After two hours, all remaining inoculated feed was removed and weighed to assure equal consumption per pen and per bird. The quantity of feed (both placed and withdrawn) was recorded on each pen's feed record.

Throughout the study, birds were observed at least three times daily for overall health, behavior, and evidence of toxicity. Pens were monitored for environmental conditions, including temperature, lighting, water, feed, litter condition, and unanticipated house conditions/events. Pens were checked daily for mortality. Examinations were performed on all broilers found dead or moribund. Mortalities were recorded (date and weight) and examined (both internal and external body mass). Throughout the study, birds were reared on built-up litter from a minimum of three previous flocks obtained from a local chicken farm to simulate stress-induced health risks related to commercial production.

Sample Collection Schedules—The studies adhered to the following collection schedules:

| Data/Sample | | | |
|---|---|---|---|
| Collected | When | Sample Size | Measurements |
| FI, BW, and mortality | Weekly | Individual weights by | FI, BW, BWG, Adjusted |
| | | sex (7, 14, 21, 28, and 42 | FCR, mortality, BW, |
| | | days) | coefficient of variation) |
| Fecal samples for: E. | Days 21 and 42 | 4 birds/pen at 21 days | Enumeration of E. |
| acervuline in loop of | | and 10 birds/pen at 42 days | acervuline in loop of |
| small intestine area, E. | | | small intestine area, E. |
| maxima in jejunum, and | | | maxima in jejunum, and |
| E. tenella in ceca. | | | E. tenella in ceca |
| Both Gut Lesion Score | Days 21 and 42 | 4 birds/pen at 21 days | Lesion scores (both |
| and Coccidia Lesion | | and 10 birds/pen at 42 days | normal gut and |
| Incidence Score of | | | coccidian lesion |
| small intestine | | | incidence score) |
| Fecal samples for: | Days 21 and 42 | 4 birds/pen at 21 days | Emeria spp. Counts |
| Digesta from small | | and 10 birds/pen at 42 | enumerated from both |
| intestine and ceca | | days | small intestine and ceca |
| Fecal samples for: | Days 21 and 42 | 4 birds/pen at 21 days | Salmonella & |
| Digesta from small | | and 10 birds/pen at 42 days | Campylobacter |
| intestine and ceca | | | incidence; E. coli, APC, |
| | | | and C. Perfringens |
| | | | enumeration |
| Villi Cell Height, Crypt | Days 21 and 42 | 4 birds/pen at 21 days | Villi Cell Height, Crypt |
| and Villus/Crypt ratio | | and 10 birds/pen at 42 days | and Villus/Crypt ratio |

Analysis Methodology

Following the multi-day treatment periods, the efficacy of the treatment compound was assessed when broiler chickens were raised under the disease challenge conditions exposed to bacteria such as but not limited to Eimeria spp and clostridium n built-up litter bedding. Overall, broiler chicken performance was assessed using various inputs, including individual body weights, feed conversion, gross necropsy results and morality over days 0-7, 0-14, 0-21, 0-28, and 0-42. Feed conversion age ranges were 0-7, 0-14, 0-21, 0-28, 0-42.

Thereafter, gross necropsy (including both external and internal measurements) was performed on birds 21 days of age for both males and females per pen and on birds 42 days of age again for both males and females. During Gross Necropsy on each bird, particular attention was given to lesion scores, Coccidiosis (small and large intestines), amount of sluffing (intestinal gut lining material that may be shown in small and large intestines), and CECA Damage Scores. Measurements and endpoints were based on growth live performance factors including mortality, feed intake, weight gains following each period and feed:gain values (feed conversion ratio), gut duodenum lesion scores and Coccidiosis/Eimeria ceca lesion score, collected fecal samples, digesta samples, and tissue samples.

To ensure statistical integrity in data evaluation, treatments were randomized by block configured into a Randomized Complete Block design. Individual chicks (half male, half female), were randomized within each block then within all treatments within 10 hours following hatch. All data points were analyzed at the 5% level of probability and included composite weighted average of entire pen, feed:gain and livability (or mortality).

Study Evaluation

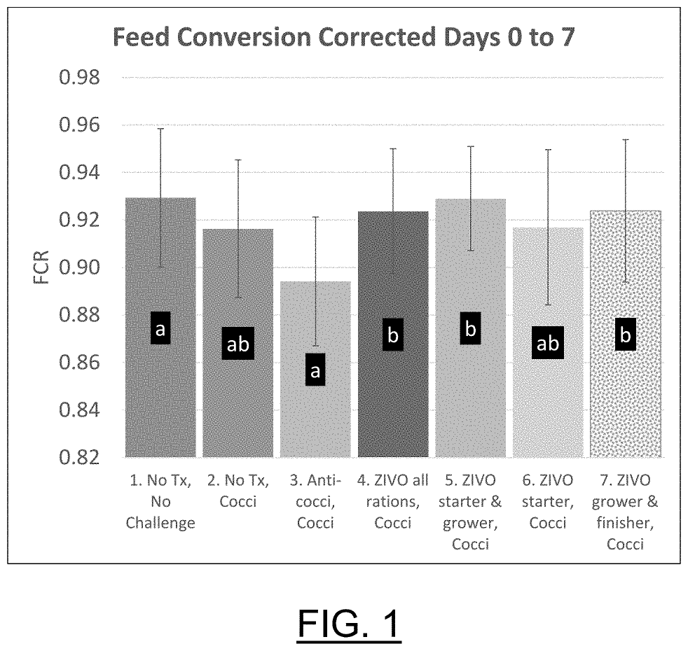

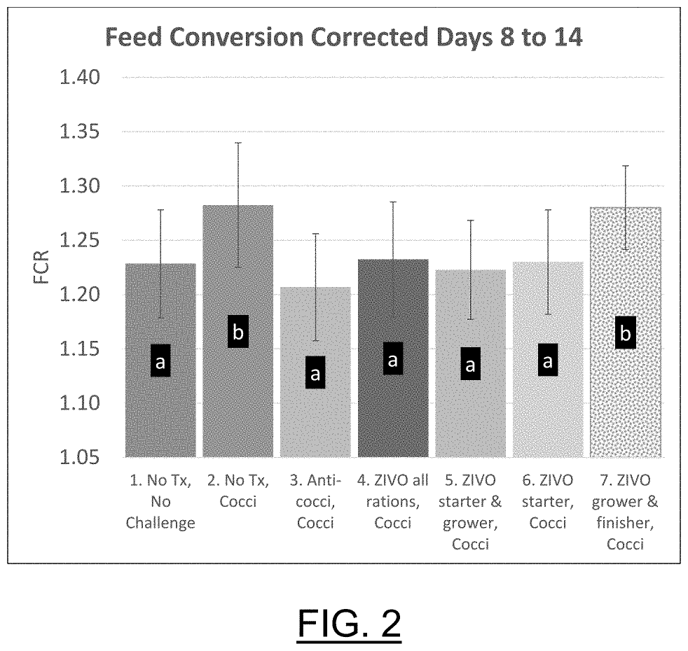

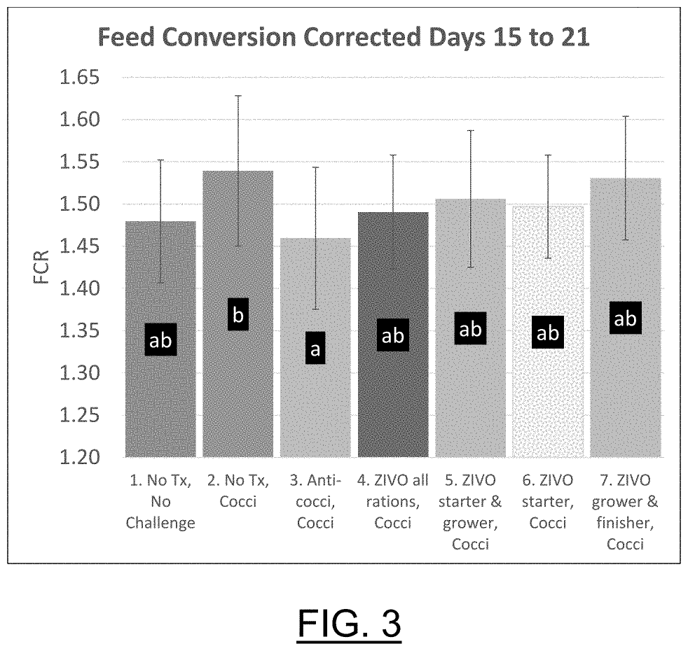

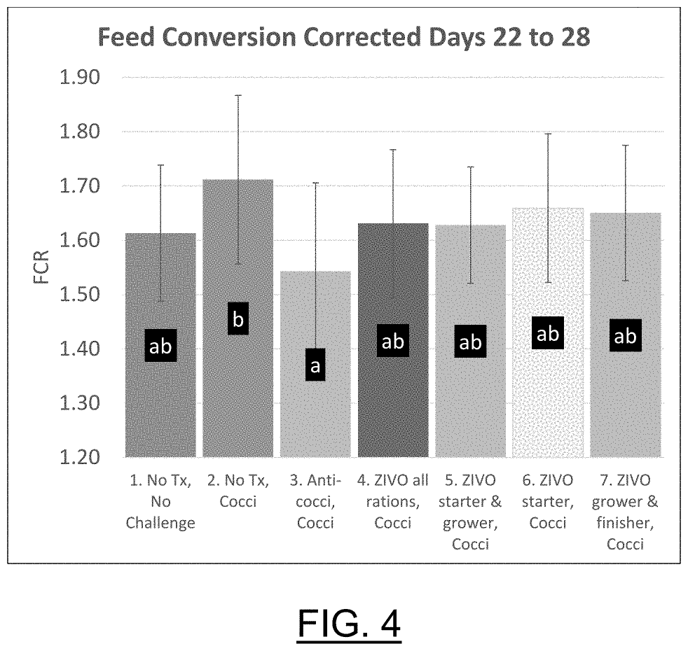

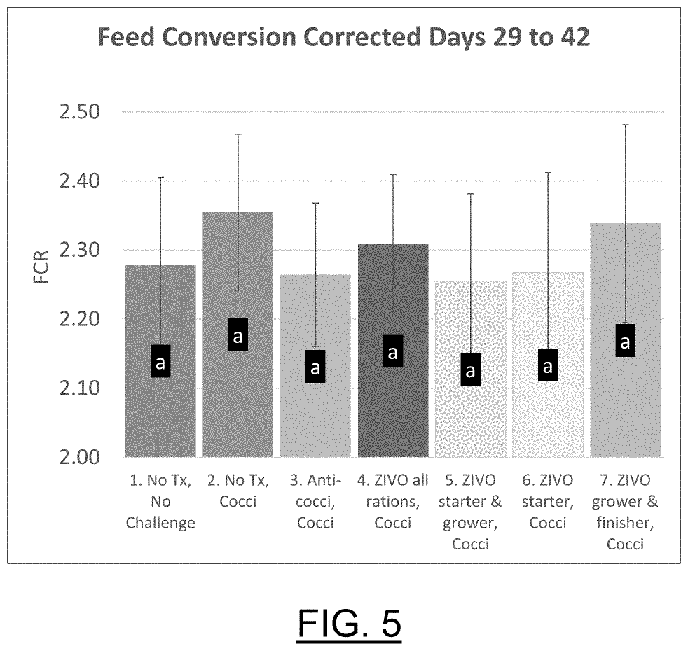

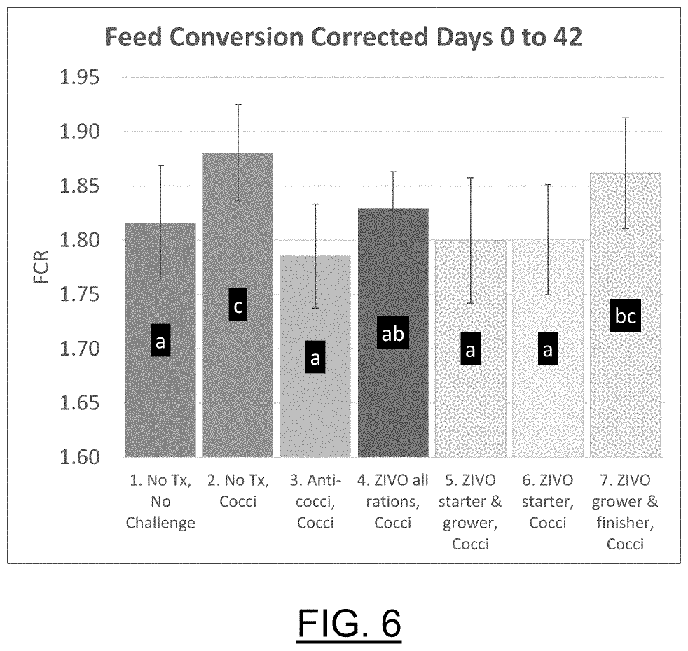

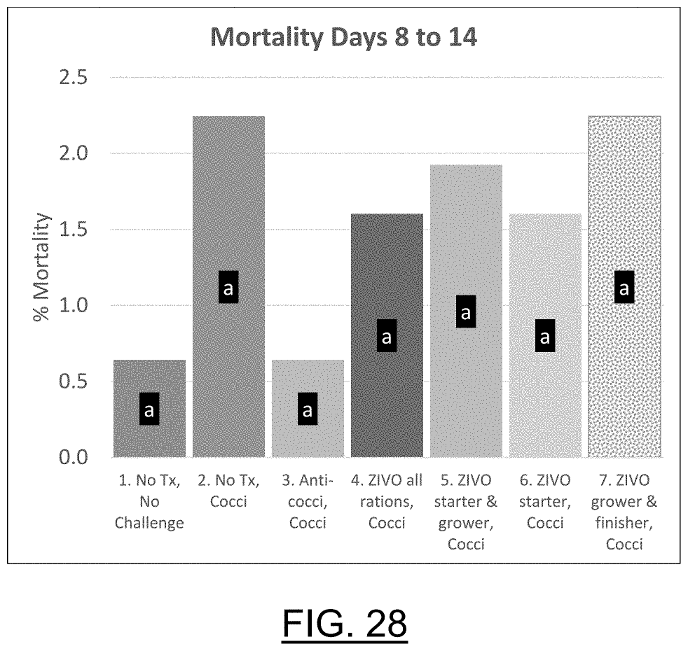

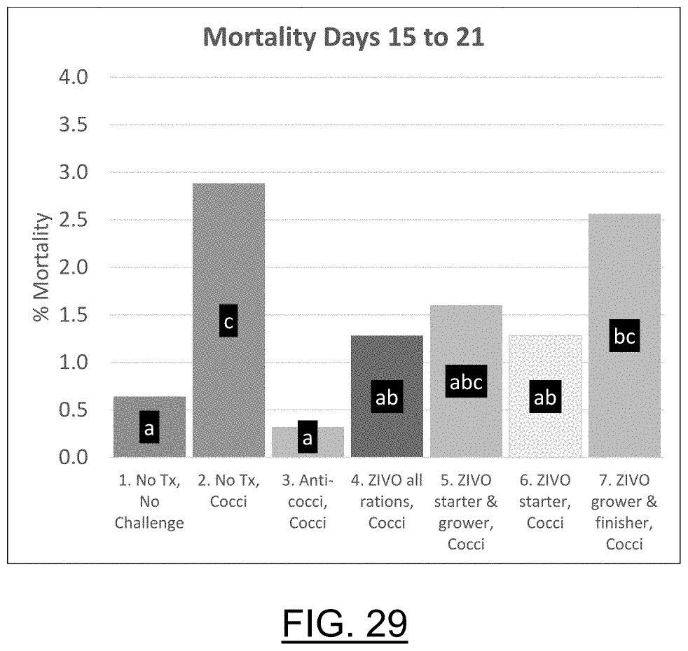

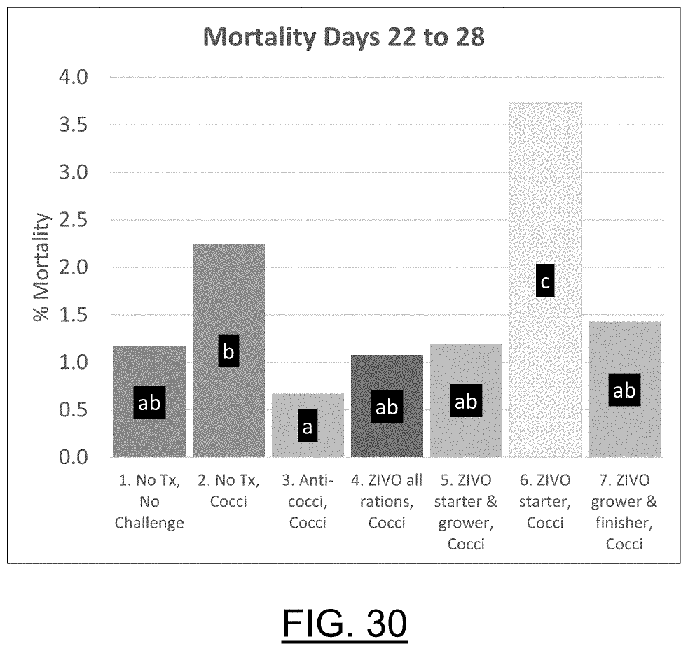

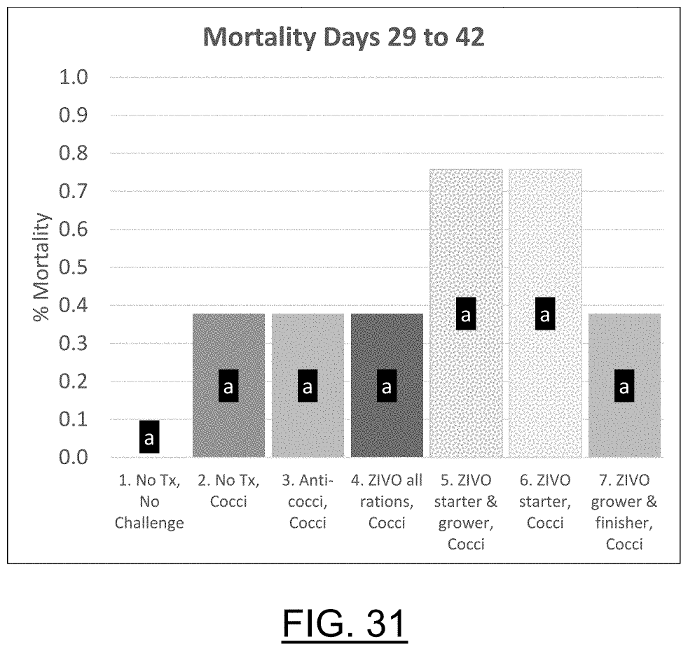

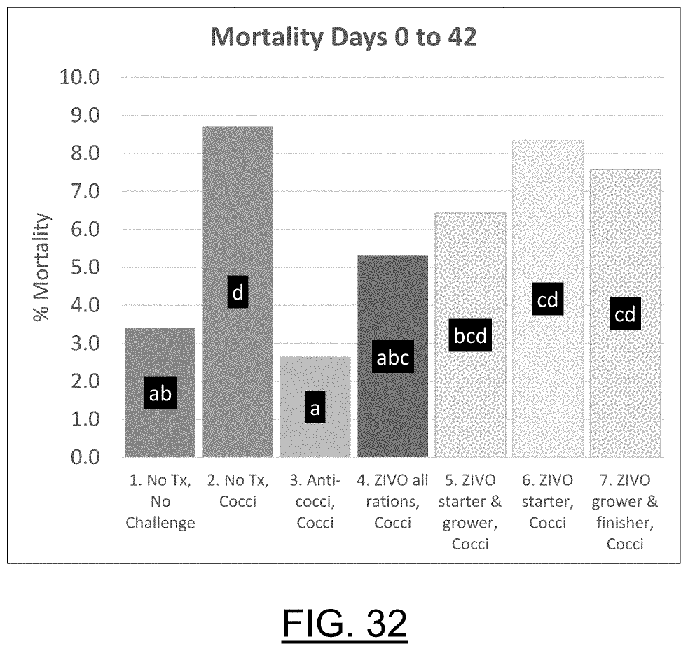

Differences between the untreated and non-diseased birds, the untreated diseased birds, the diseased birds treated with a conventional antibiotic over various periods of time between 0 and 42 days, and the diseased birds treated with different inventive compounds are illustrated in the graphs shown in FIGS. 1 through 32. The graphs are directed to feed conversion ratios (FCRs), lesion scores, ileum villi cell height, ileum crypt depth, ileum cell height to crypt depth ratio, various fecal counts (Salmonella, Clostridium perfringens, and E. coli), duodenum loop oocyst counts, mid-gut oocyst counts, whole cecum oocyst counts, and mortality.

Feed Conversion—The feed conversion rate (FCR) is a useful indicator of how efficiently an animal uses feed. Animals demonstrating a low FCR are generally regarded as efficient users of feed. A low FCR also suggests a good quality feed.

As illustrated in FIGS. 1 through 6, mortality-corrected Feed Conversion Ratio was measured and reported for Days 0-7, 8-14, 15-21, 22-28, 29-42, and 0-42. With the exception of Days 0-7, the disclosed inventive compounds consistently provided improved results when compared with the untreated and coccidiosis-diseased group. Most notable are the positive results achieved by the application of the ZIVO starter & grower and starter feeds at Days 29-42 when both feeds demonstrated an FCR advantage over coccidiosis-diseased birds treated with conventional anti-coccidiosis treatment.

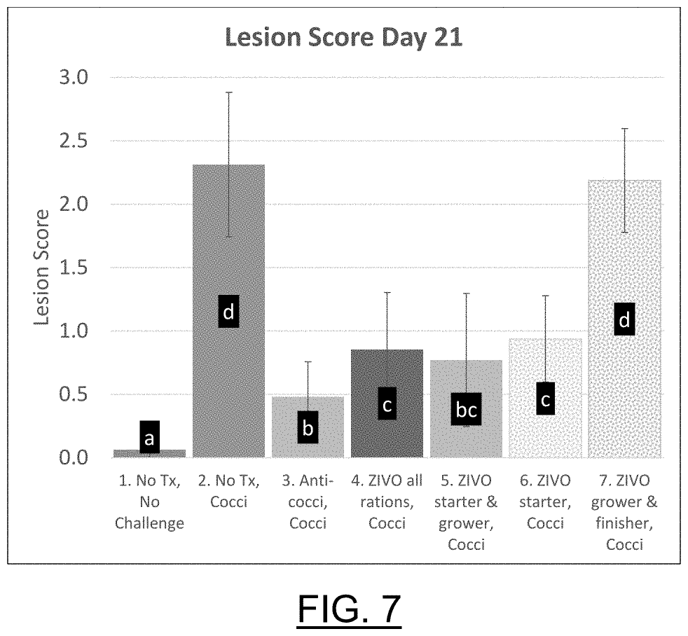

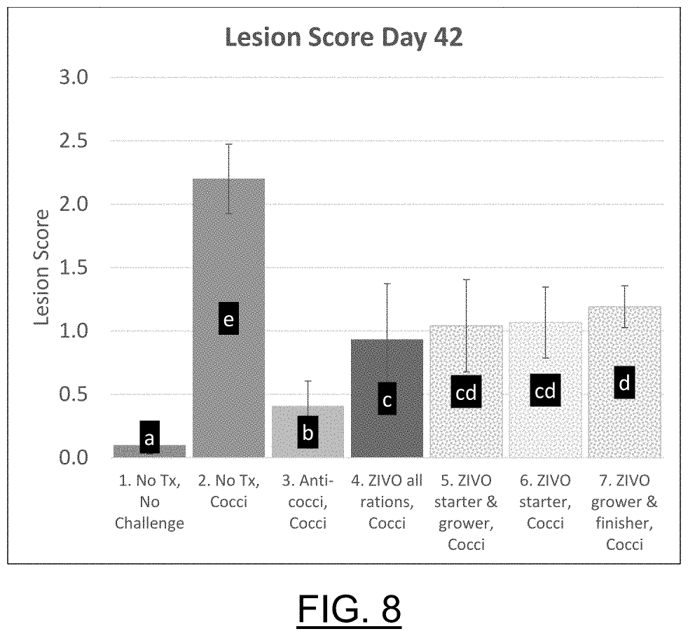

Lesion Scoring—Gross necropsy and lesion scoring were performed on Days 21 and 42. Birds were selected, sacrificed, weighed, and examined for the presence and degree of coccidia lesions and the amount of intestinal gut lining sluffing. CECA damage scores were assessed and recorded as illustrated in FIGS. 7 and 8. By Day 42, the lesion score was significantly reduced across all groups treated with all ZIVO formulations and consistently showed advantage over the non-treated birds.

ILEUM VILLI CHARACTERISTICS—Ileum villi are important structures found in the small intestine. The villi are involved mainly in nutrient absorption and, accordingly, the increase of villi height and consequent increase in absorptive surface area affects the nutrient absorption capability in the intestine.

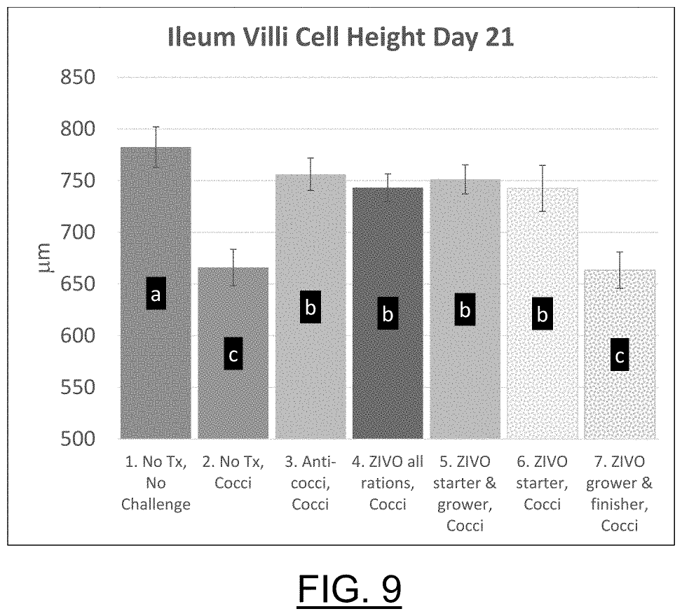

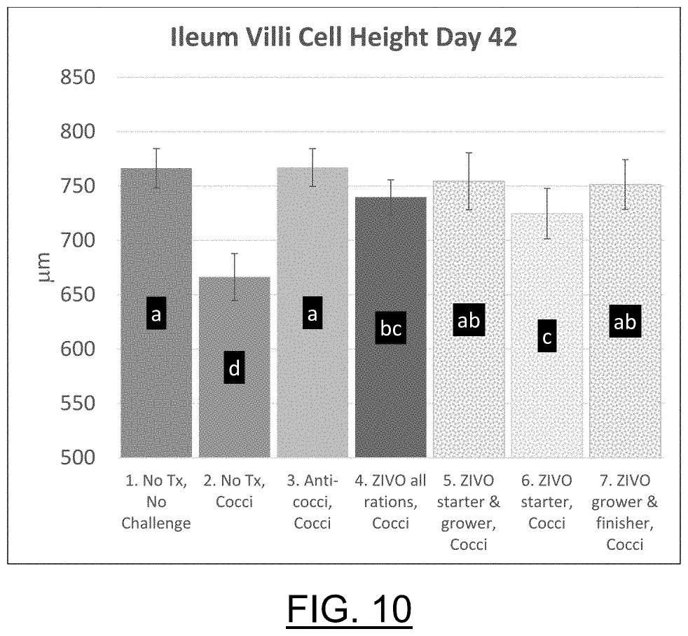

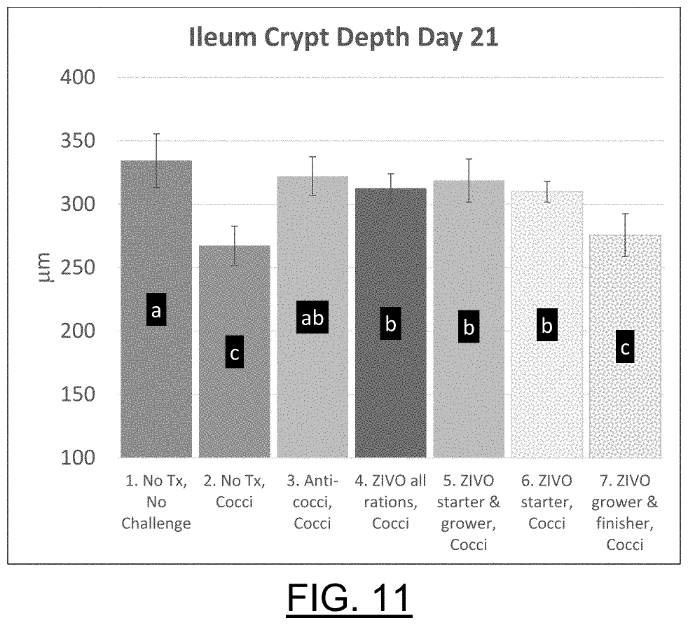

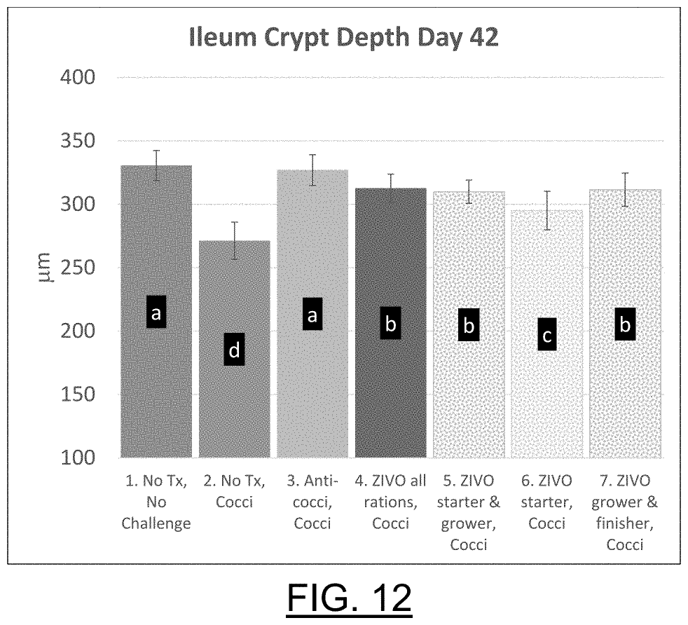

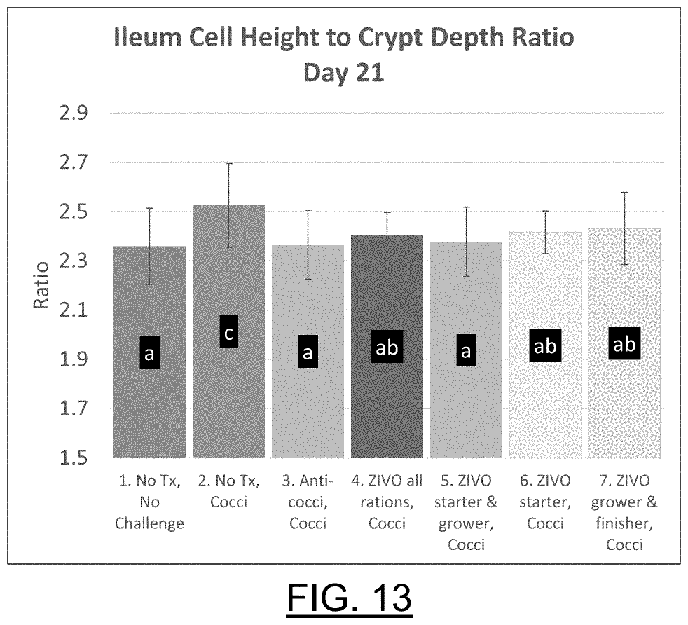

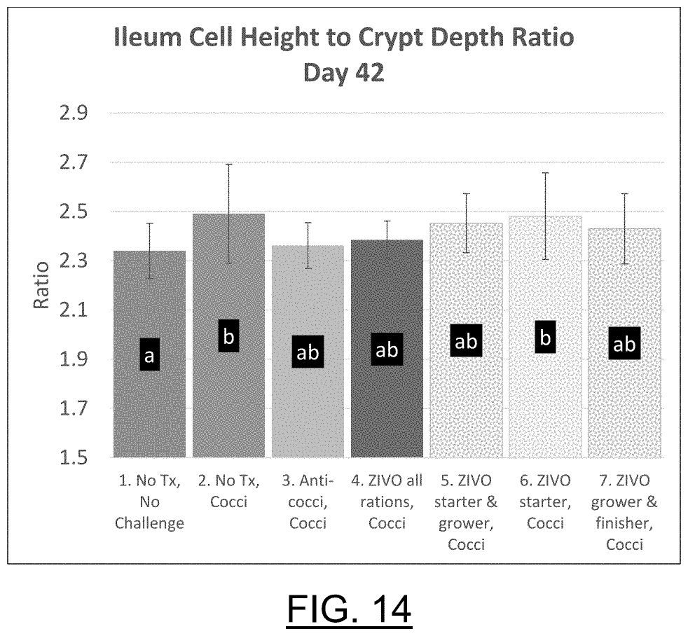

Ileum villi cell height, crypt depth, and the villus height to crypt depth ratio were taken, calculated and reported from the ileum area on Days 21 and 42. Referring to FIGS. 9 and 10, ileum villi cell height remained generally steady for all ZIVO formulations between Days 21 and 42 with the exception of the grower & finisher which showed significant improvement by Day 42. Importantly, ileum villi cell height in birds fed the ZIVO formulations was competitive with coccidiosis-challenged birds treated with conventional antibiotics. Similar results were seen with ileum crypt depth between Days 21 and 42 as illustrated in FIGS. 11 and 12. Significantly, by Day 42 the ileum cell height to crypt depth ratio showed improvements for birds fed with all variations of the ZIVO feed as illustrated in FIGS. 13 and 14.

As shown, the ileum villi height-to-depth ratio of the coccidia-challenged animals demonstrates the morphological results of a compromised ability to absorb nutrients brought on by disease. The remaining conditions demonstrate a healthy gut having a relatively high absorptive surface area for nutrients with the birds having been treated with the disclosed treatment compound showing a villi height-to-depth ratio almost the same as those without being subject to the coccidia challenge as well as those to which an anti-coccidiosis treatment was administered but without the accompanying side effects.

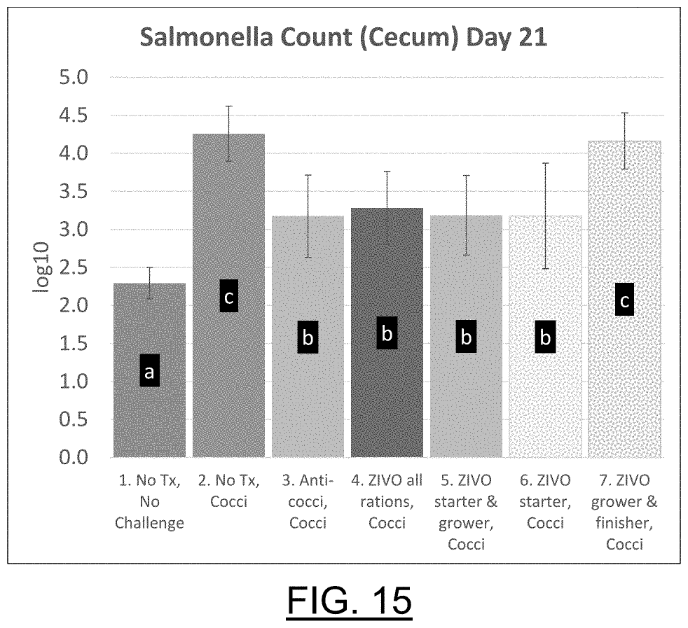

Bacteria—As noted above, coccidiosis damages the gut of the animal, thus often acting as a predisposing factor to the rapid onset of bacterial infection and consequential disease, such as necrotic enteritis. Poultry are susceptible to various bacteria, including Salmonella, C. perfringens, and E. coli. As illustrated in FIGS. 15 through 20, samples from the cecum as well as from feces were evaluated for the presence of bacteria on both Day 21 and Day 42. The intestinal and fecal samples were analyzed to determine a total aerobic plate count (APC).

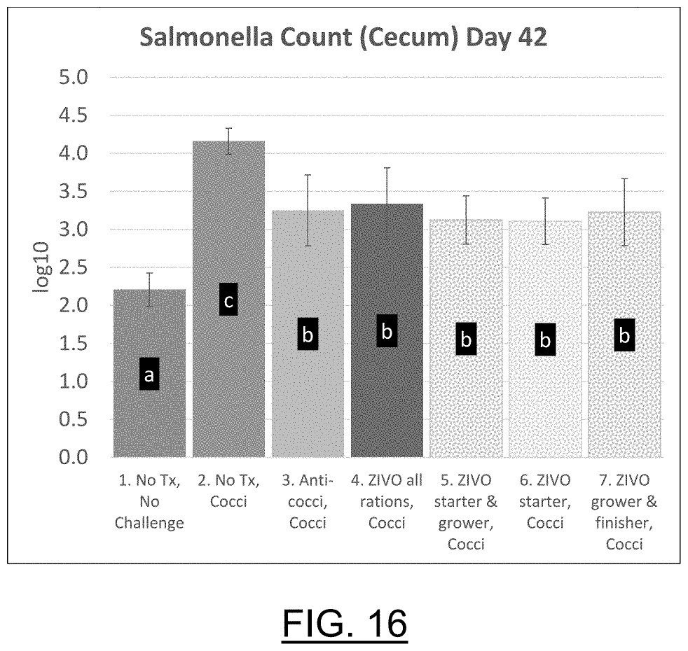

With respect to data related to Salmonella, FIGS. 15 and 16 illustrate the differences between Day 21 and 42 in which it can be seen that the cecum count generally dropped for birds fed the ZIVO feed variations by Day 42 illustrating the latent benefit of the disclosed composition. Conversely, the count rose over the same period for coccidia-challenged birds subjected to conventional anti-coccidiosis treatment

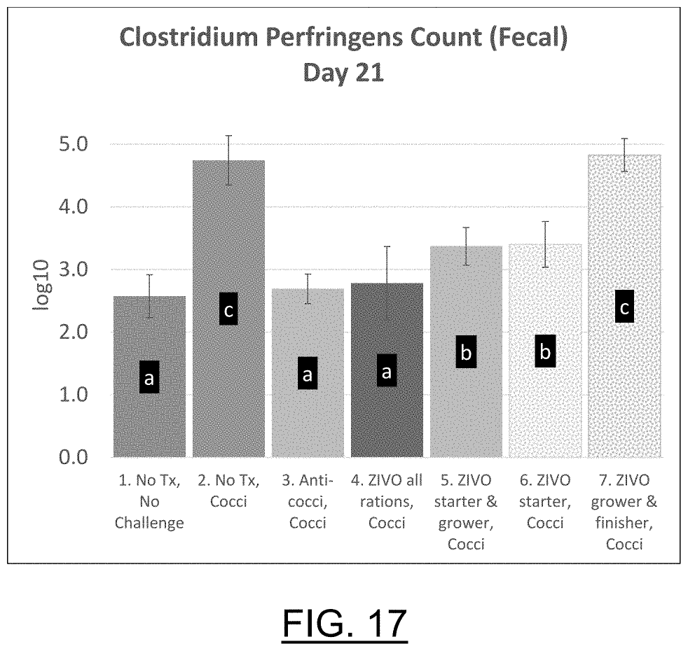

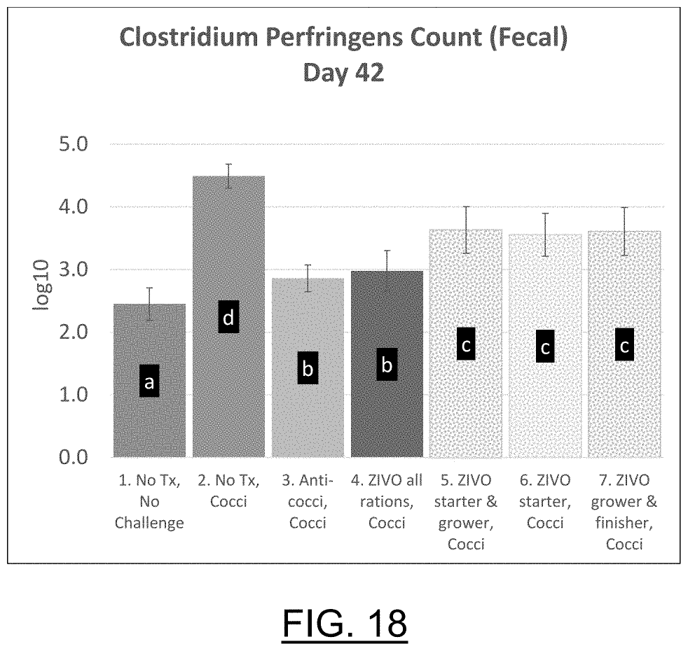

With respect to data related to C. perfringens, FIGS. 17 and 18 illustrate the differences between Day 21 and 42 in which it can be seen that the fecal count generally rose slightly for birds fed the “ZIVO starter & grower” and “ZIVO starter” feed but showed a relatively dramatic decrease in birds fed the “ZIVO grower & finisher” composition by Day 42 underscoring the long-term benefit of the present composition.

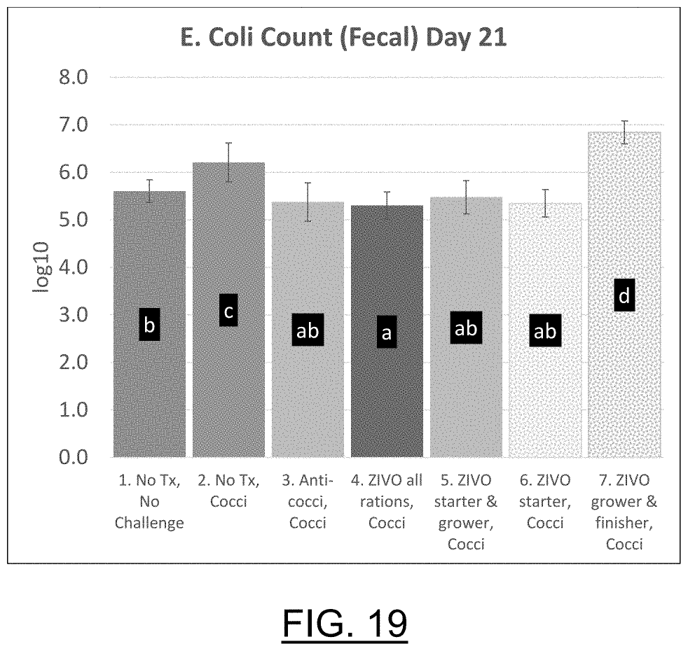

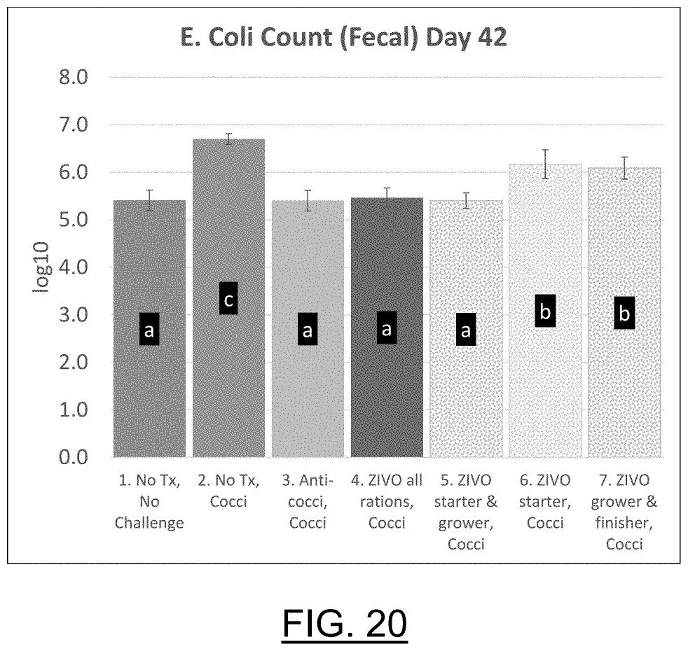

With respect to data related to E. coli, FIGS. 19 and 20 illustrate the differences between Day 21 and 42 in which it can be seen that by Day 42 the fecal count dropped slightly in animals fed the “ZIVO starter & grower” composition, rose for birds fed the “ZIVO starter” feed, and dramatically decreased in birds fed the “ZIVO grower & finisher” composition.

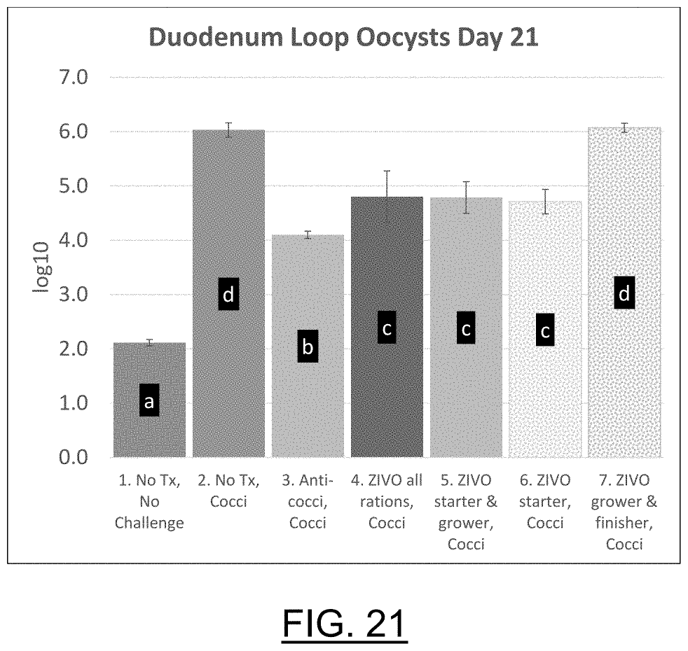

Oocyst Score—Gross necropsy and oocyst scoring were performed on Days 21 and 42 at different locations on the animal. Previously oocyst-inoculated birds were selected, sacrificed, weighed, and examined for the presence and degree of oocysts in their duodenum loop, mid-gut, and whole cecum. The results of the study are illustrated in FIGS. 21 through 26.

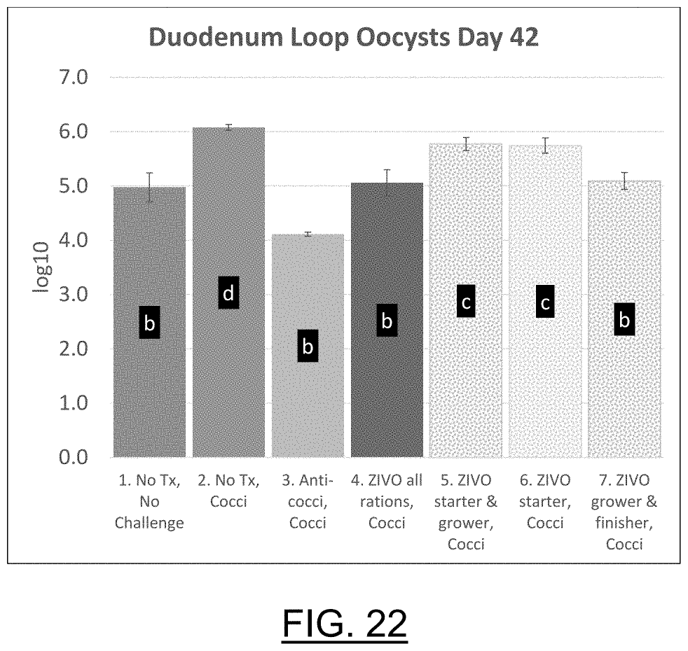

With respect to the duodenum loop oocyst counts for Days 21 and 42 of FIGS. 21 and 22 respectively, by Day 42 the duodenum loop oocyst count generally rose both for birds fed the “ZIVO starter & grower” composition and the “ZIVO starter” composition but fell significantly for birds fed the “ZIVO grower & finisher” composition.

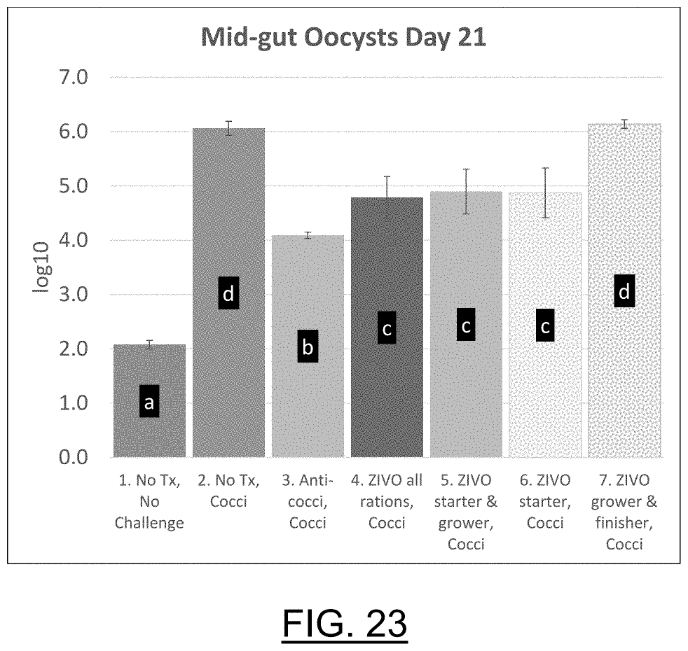

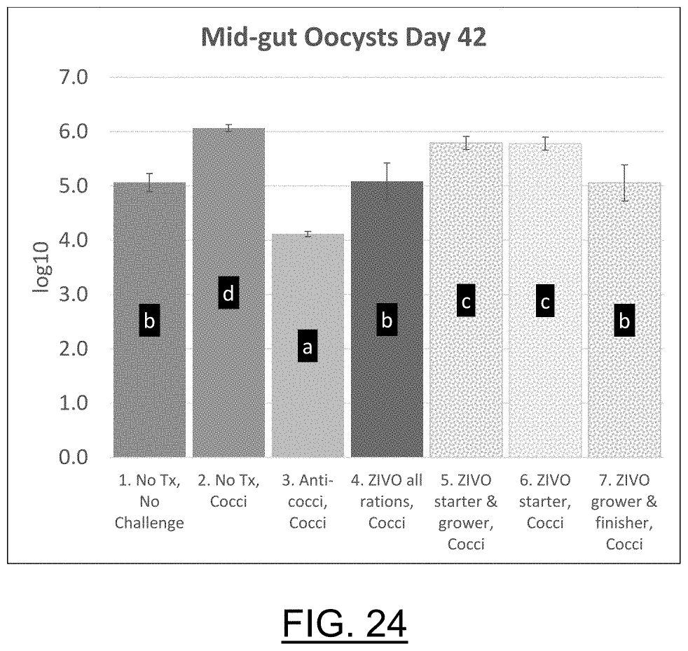

With respect to the mid-gut oocyst counts for Days 21 and 42 of FIGS. 23 and 24 respectively, by Day 42 the oocyst count generally rose both for birds fed the “ZIVO starter & grower” composition and the “ZIVO starter” composition but fell for birds fed the “ZIVO grower & finisher” composition.

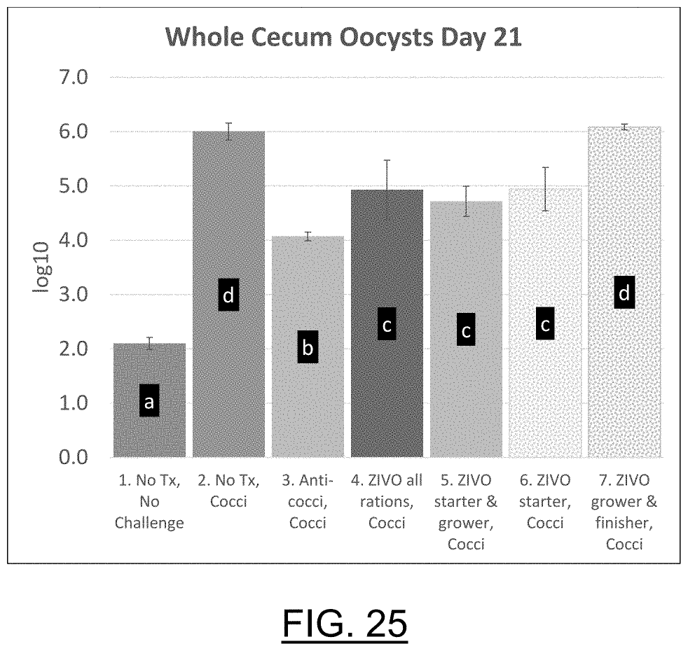

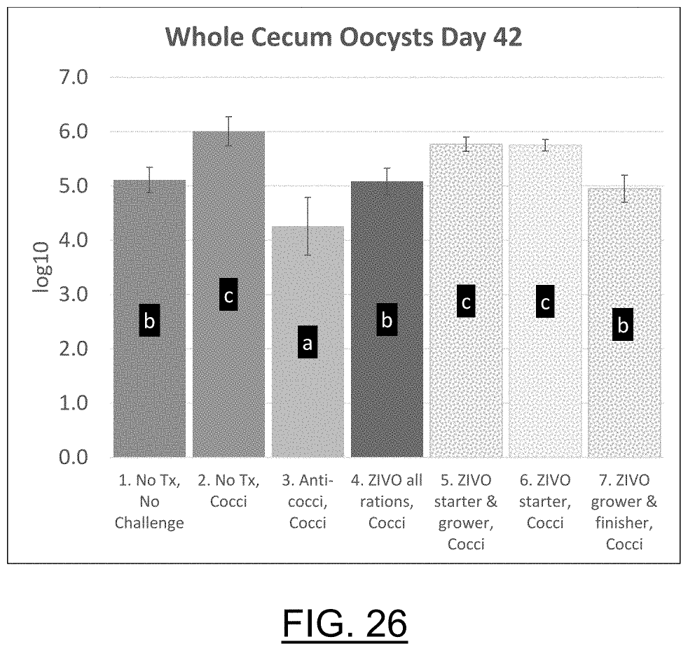

With respect to the whole cecum oocyst counts for Days 21 and 42 of FIGS. 25 and 26 respectively, by Day 42 the oocyst count generally rose both for birds fed the “ZIVO starter & grower” composition and the “ZIVO starter” composition but fell for birds fed the “ZIVO grower & finisher” composition.

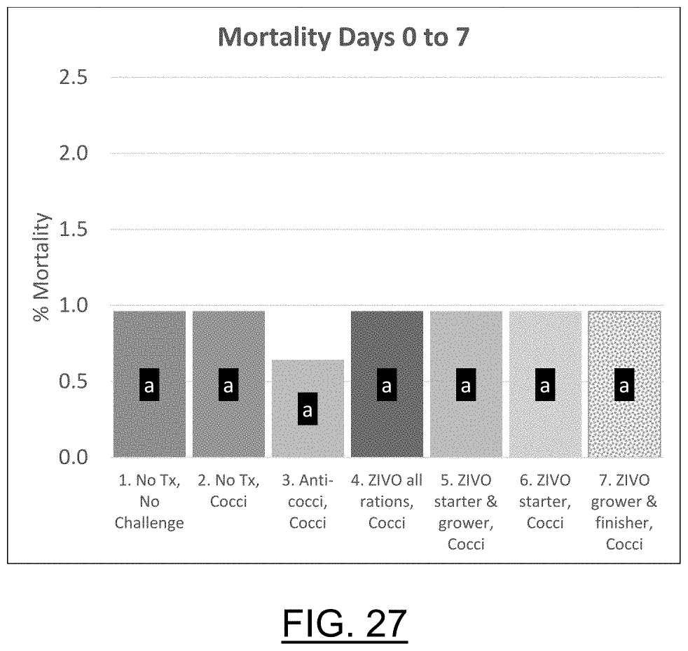

Mortality—As illustrated in FIGS. 27 through 32, mortality was calculated for Days 0-7, 8-14, 15-21, 22-28, 29-42, and 0-42. Across all age periods, the % mortality of the untreated and diseased group was normally but not always higher than for the groups given feed containing the various ZIVO compositions.

Results

In general, analysis of the results supports the conclusion that use of the innovative compound in the treatment of coccidiosis-challenged poultry demonstrates a positive latency effect on the coccidiosis prevention and treatment by delivery through animal feed compared with coccidiosis-challenged and untreated poultry. The positive results noted below were identified in the different bacterial variations of the composition of the disclosed inventive concept.

Kinomic analysis of the broiler tissues from both groups of chickens fed the treatment compound according to the regimen set forth above as well as those not fed the treatment compound verified that the treated chickens demonstrated an altered immune response consistent with the effects of TLR4 inhibition. Overall, kinomics analysis of birds fed the test material for the first 14 days of life demonstrated changes in the immune system consistent with a bolstered innate immune response thereby providing positive latency effects over the life of the animal.

Notably, a significant decrease in the presence and degree of coccidia lesions and damage to the intestinal lining typically experienced following coccidiosis infection was noted. It is evident that the disclosed treatment method and composition, acting as a non-antibiotic alternative, demonstrates a significant decrease in the presence of pathogenic bacteria in the gut, such as salmonella, Clostridium perfringens, and E. coli, which are naturally occurring microbes in poultry production.

The specific results are summarized as follows:

The FCR showed improvement in the sample poultry fed the disclosed composition compared with untreated disease-challenged birds.

Upon examination of sacrificed sample birds, it was found that the average lesion scores of both the duodenum and the ceca of sample poultry treated with the disclosed composition were consistently lower than the scores of sacrificed untreated disease-challenged birds. Feed composition having the “ZIVO grower & finisher” product significantly aided in reducing lesion scores by Day 42.

Ileum villi cell height, crypt depth, and the villus height to crypt depth ratio generally showed improvement or at remained steady over time. Particularly, ileum villi cell height and crypt depths in birds fed the ZIVO formulations were competitive with coccidiosis-challenged birds treated with conventional antibiotics.

It was found that the presence of various bacteria, including Salmonella, C. perfringens, and E. coli, was generally reduced in birds fed a ZIVO composition in their feed compared with untreated birds.

Upon examination of sacrificed sample birds, it was found that the average oocyst count of the duodenum, mid-gut, and cecum of sample poultry given feed having a ZIVO composition were lower than the scores of sacrificed untreated disease-challenged birds.

Mortality—The % mortality of the untreated and diseased group was normally but not always higher than for the groups given feed containing the various ZIVO compositions. The data confirm the positive effect of the ZIVO formulations on the study birds.

The improvement of the overall health of disease-challenged poultry as a result of being given feed with the disclosed inventive composition was achieved without the use of antibiotics.

Overall the inventive composition demonstrates a cost-effective and practical approach to the treatment of disease states in animals.

What is claimed is:

1. A method for preventing or minimizing coccidial the risk of infection in an animal substantially during its lifetime, the method comprising feeding to the animal an effective amount of a composition comprising a lipopolysaccharide derived from Gram-negative bacteria, the method including the step of initially feeding the animal the effective amount of the composition during the first week of life.

2. The method of claim 1, the method including the step of monitoring the animal for the presence of coccidial infection or for cecal lesions after the initial feeding.

3. The method of claim 1, whereby the composition is mixed with a feed ration portion prior to feeding the animal.

4. The method of claim 3, wherein the composition comprising the lipopolysaccharide derived from Gram-negative bacteria is fed to the animal in an amount providing from about 0.5 lbs. composition per ton of finished feed to about 11.0 lbs. composition per ton of finished feed.

5. The method of claim 3, wherein the composition comprising the lipopolysaccharide derived from Gram-negative bacteria is fed to the animal in an amount providing from about 1.0 lbs. composition per ton of finished feed to about 5.0 lbs. composition per ton of finished feed.

6. The method of claim 3, wherein the composition comprising the lipopolysaccharide derived from Gram-negative bacteria is fed to the animal in an amount providing from about 3.0 lbs. composition per ton of finished feed to about 4.0 lbs. composition per ton of finished feed.

7. The method of claim 1, wherein the composition comprising the lipopolysaccharide derived from Gram-negative bacteria is formulated for feeding to bovine, porcine, avian, equine, ovine, lapine, and caprine species.

8. The method of claim 1 wherein said Gram-negative bacteria is a member of the group Variovorax.

9. The method of claim 8 wherein said member of the group Variovorax is Variovorax paradoxus.

10. The method of claim 1 wherein the composition comprising the lipopolysaccharide derived from Gram-negative bacteria composition is for the prevention and treatment of coccidiosis in poultry.

11. A method of protecting a recipient against coccidiosis, comprising administering to the recipient feed including a non-antibiotic composition in the form of a lipopolysaccharide derived from Gram-negative bacteria in an amount effective to minimize the risk of the animal becoming infected with coccidiosis, the composition being fed to the animal in an amount providing from about 0.5 lbs. composition per ton of finished feed to about 11.0 lbs. composition per ton of finished feed.

12. The method of claim 11, wherein the composition comprising the lipopolysaccharide derived from Gram-negative bacteria is fed to the animal in an amount providing from about 1.0 lbs. composition per ton of finished feed to about 5.0 lbs. composition per ton of finished feed.

13. The method of claim 11, wherein the composition comprising the lipopolysaccharide derived from Gram-negative bacteria is fed to the animal in an amount providing from about 3.0 lbs. composition per ton of finished feed to about 4.0 lbs. composition per ton of finished feed.

14. The method of claim 11, wherein the composition comprising the lipopolysaccharide derived from Gram-negative bacteria is formulated for feeding to bovine, porcine, avian, equine, ovine, lapine, and caprine species.

15. The method of claim 11, wherein said Gram-negative bacteria is a member of the group Variovorax.

16. The method of claim 15, wherein said member of the group Variovorax is Variovorax paradoxus.

17. The method of claim 11, wherein the composition comprising the lipopolysaccharide derived from Gram-negative bacteria composition is for the prevention and treatment of coccidiosis in poultry.

18. A method for preventing or minimizing coccidial the risk of infection in an animal substantially during its lifetime, the method comprising feeding to the animal an effective amount of a composition comprising a lipopolysaccharide derived from Gram-negative bacteria, the method including the step of feeding the animal the effective amount of the composition during the first week of life, whereby subsequent treatments of the composition beyond the first week of life are rendered unnecessary in the prevention of coccodia infection.

19. The method of claim 18, wherein the composition comprising the lipopolysaccharide derived from Gram-negative bacteria is fed to the animal in an amount providing from about 0.5 lbs. composition per ton of finished feed to about 11.0 lbs. composition per ton of finished feed.

20. The method of claim 18, wherein the composition comprising the lipopolysaccharide derived from Gram-negative bacteria is fed to the animal in an amount providing from about 1.0 lbs. composition per ton of finished feed to about 5.0 lbs. composition per ton of finished feed.

21. The method of claim 18, wherein the composition comprising the lipopolysaccharide derived from Gram-negative bacteria is fed to the animal in an amount providing from about 3.0 lbs. composition per ton of finished feed to about 4.0 lbs. composition per ton of finished feed.

22. A composition for the treatment of coccidiosis in animals, the composition comprising effective amounts of a feed ingredient is a biomass selected from the group consisting of an algal biomass and a bacterial biomass.

23. The composition of claim 22 wherein the biomass is a bacterial biomass including a lipopolysaccharide derived from Gram-negative bacteria.

24. The composition of claim 22 wherein the biomass is a bacterial biomass including one of a supernatant, a symbiont bacteria, or bacterial fermentate.
